(19) United States
(12) Patent Application Publication
(10) Pub. No.: US 2025/0127617 A1
(43) Pub. Date: Apr. 24, 2025

(54) DELIVERY SYSTEM FOR PLACEMENT OF ENDOLUMINAL DEVICES

(71) Applicant: Frond Medical Inc., Fremont, CA (US)

(72) Inventors: Xin Lu, Palo Alto, CA (US); Rose Monaghan, Los Gatos, CA (US); Arnold Seto, Long Beach, CA (US)

(21) Appl. No.: 18/905,753

(22) Filed: Oct. 3, 2024

Related U.S. Application Data
(60) Provisional application No. 63/588,088, filed on Oct. 5, 2023.
(60) Provisional application No. 63/595,606, filed on Nov. 2, 2023.
(60) Provisional application No. 63/600,960, filed on Nov. 20, 2023.

(51) Int. Cl. A61F 2/24; A61B 5/00; A61B 8/12; A61M 25/09

(52) U.S. Cl. CPC A61F 2/2433; A61B 5/0066; A61B 5/0084; A61B 8/12; A61M 25/09

(57) ABSTRACT

Systems and methods for the placement of an endoluminal device are provided. A delivery system for endoluminal devices includes a delivery catheter comprising an imaging sensor and a position sensor. The position sensor is located at a known distance from the imaging sensor and configured to sense relative position to an element with a position code that is readable by the position sensor. The delivery catheter is configured for mounting an endoluminal device on the delivery catheter at a known distance from the imaging sensor or position sensor. The imaging sensor, the position sensor, or both the imaging sensor and the position sensor can be used for intravascular imaging of an anatomical location, guidance for deployment of the endoluminal device, confirmation of the placement of the endoluminal device at the anatomical location, or any combination thereof.

### RELATED APPLICATIONS

This application claims the benefit of U.S. Provisional Application No. 63/588,088, filed on Oct. 5, 2023, U.S. Provisional Application No. 63/595,606, filed on Nov. 2, 2023, and U.S. Provisional Application No. 63/600,960, filed Nov. 20, 2023.

The entire teachings of the above applications are incorporated herein by reference.

### BACKGROUND

Endovascular interventional therapies often require accurate deployment of a catheter-mounted endoluminal devices, for example an aortic valve, a stent graft, or a stent, within a body lumen. Currently available methods for guiding and positioning the catheter are based upon real-time X-ray angiographic imaging, which can result in high dosages of radiation exposure and contrast agents to a patient. Transesophageal echocardiogram can provide non-radiographic imaging guidance for some procedures, including transcatheter aortic valve replacement (TAVR), but can require the use of general anesthesia and endotracheal intubation and is limited by contraindications against esophageal or gastric procedures.

Intravascular imaging sensors, including intravascular ultrasound (IVUS) and optical coherence tomography (OCT), can provide guidance near the treatment site. However, these modalities typically require displacing the diagnostic sensor through a body lumen length to generate body lumen information at closely spaced displacement points. This process can be actuated by a motor unit external to a patient, but motor position readings external to the patient may not be accurately reflective of sensor position displacement within the body lumen due to reasons including the inherent elasticity of physiological vessels and tissues. Therapeutic intervention can then further require precise re-traversal along an imaged body lumen to a treatment site imaged by the intravascular imaging sensors.

There exists a need for improved systems and methods for perioperatively guiding the deployment of endoluminal devices and locating medical devices within a body lumen during interventional procedures.

### SUMMARY

Systems and methods are provided for the placement of endoluminal devices utilizing a position sensor and an element with position codes readable by the position sensor, an imaging sensor, radiopaque imaging markers, or a combination thereof. The systems and methods described are useful for various endoluminal procedures, including cardiovascular valve replacement and aneurism repair.

An example delivery system for endoluminal devices includes a delivery catheter comprising an imaging sensor and a position sensor. The position sensor is located at a known distance from the imaging sensor and configured to sense relative position to an element with a position code that is readable by the position sensor. The delivery catheter is configured for mounting an endoluminal device on the delivery catheter at a known distance from the imaging sensor or the position sensor.

The imaging sensor can be an intravascular ultrasound (IVUS) sensor or an optical coherence tomography (OCT) sensor. The imaging sensor can be located between the endoluminal device and a distal end of the delivery catheter.

The element with the position code can be a guidewire, with the position sensor configured to read the position code from the guidewire. The system can include the element with the position code. The delivery catheter can include a lumen for receiving the guidewire and the lumen can extend through the imaging sensor and the position sensor.

The position sensor configured to read the position code can be located between the endoluminal device and a proximal end of the delivery catheter. Alternatively, the position sensor can be positioned near the distal end of the delivery catheter.

The endoluminal device can be, for example, an aortic valve, a stent graft, or a stent. The endoluminal devices can also include one or more of an ultrasound heating device, an acoustic emitting device, a heating device, or a vessel cutting device.

The system can include a deployment mechanism to deploy the endoluminal device in a body lumen, such as a balloon-expandable endoluminal device with a balloon membrane deployment mechanism. The imaging sensor of the system can be positioned inside the balloon membrane. Further, the system can include a handle at a proximal end of the delivery catheter configured to activate the deployment mechanism. The position sensor configured to read the position code of the element can be positioned in the handle.

The system can include the endoluminal device, which can be mounted on the delivery catheter at a known distance from the imaging sensor or the position sensor.

The delivery catheter can include an outer sheath and an inner shaft that is movable relative to the outer sheath. The outer sheath and inner shaft can be configured such that one of the inner shaft or outer sheath is the element with the position code and the other includes the position sensor. The imaging sensor can be positioned at or on the inner shaft. The imaging sensor can be an IVUS transducer configured to detachably mount to the inner shaft.

The imaging sensor can be configured to have side-looking or forward-looking capabilities. The imaging sensor can include two imaging sensors, for example two IVUS ring array transducers, with at least one of the ring array transducers configured to have forward-looking capability.

The element with the position code can be a guidewire, an inner shaft of the delivery catheter, or an outer sheath of the delivery catheter. The position sensor can be one or more of an optical, electrical, electromagnetic, mechanical, electrochemical, pressure, chemically-selective sensor, or sonographic sensor.

The delivery system, e.g., the delivery catheter, can include one or more imaging markers, e.g., radiopaque marker or other imaging makers viewable by an imaging system. Spatial alignment of the position code with respect to the one or more imaging markers can enable registration of a coordinate frame of reference of the delivery system with a frame of reference of an imaging system.

A method for the placement of an endoluminal device using a delivery system includes: 1) guiding a delivery catheter and endoluminal device mounted on the delivery catheter through a body lumen to an anatomical location, 2) placing the endoluminal device at the anatomical location, and 3) confirming placement of the endoluminal device at the anatomical location with an imaging sensor, position sensor, or both an imaging sensor and a position sensor. The position sensor can be located at a known distance from the imaging sensor and configured to sense position relative to an element with position code readable by the position sensor. The method can further include mounting the endoluminal device on the delivery catheter at a known distance from the imaging sensor or position sensor.

A method for the placement of an aortic valve includes: 1) guiding a delivery catheter that includes an imaging sensor, a position sensor, and an aortic valve, mounted on the catheter and located at a known distance from the imaging sensor or the position sensor, to the heart; and 2) placing the aortic valve in the heart at a location confirmed by the imaging sensor, the position sensor, or both the imaging sensor and the position sensor.

The position sensor can be located at a known distance from the imaging sensor and configured to sense position relative to an element with a position code readable by the position sensor. The element can be a guidewire, an inner shaft of the delivery catheter, or an outer sheath of the delivery catheter. The position sensor can be located between the aortic valve and a proximal end of the delivery catheter.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A illustrates an example delivery system for placement of an endoluminal device with a balloon membrane.

FIG. 1B illustrates the delivery system of FIG. 1A with the endoluminal device expanded by expansion of a balloon membrane.

FIG. 2 illustrates an example delivery system with a handle and a position sensor located on the delivery catheter.

FIG. 3A illustrates an example delivery system including a delivery catheter with a position code, position sensor, and imaging sensor, with the imaging sensor positioned at or on an inner shaft of a delivery catheter.

FIG. 3B illustrates an example delivery system including a delivery catheter with a position code, position sensor, and imaging sensor, with the imaging sensor positioned near a distal tip of the delivery catheter.

FIG. 4 illustrates an example imaging sensor including two imaging ring array sensors, with at least one ring array sensor configured to fire in a forward-facing direction.

FIG. 5 illustrates an example delivery system with a handle and a position sensor located at or on the handle.

### DETAILED DESCRIPTION

A description of example embodiments follows.

Systems and methods for placement of an endoluminal device are described. Embodiments are useful for various endoluminal procedures, including valve replacement and aneurism repair, among others.

To replace an aortic valve, a procedure called Transcatheter Aortic Valve Replacement (TAVR) can be used to deliver and place an artificial valve in the heart using a catheter. The artificial valve is compressed and mounted onto the catheter in a compressed state. To access the heart, the clinician makes a small incision in an artery or other blood vessel, most often in the groin, and inserts the catheter and compressed valve. Typically, a guidewire is inserted and extended through the blood vessel to the heart. The catheter and compressed valve can travel along the guidewire and through a blood vessel until they reach the diseased aortic valve. The clinician then expands the artificial valve, thereby pushing the diseased parts of the aortic valve leaflets out of the way. During the procedure, visualization via X-ray imaging is typically used to guide the positioning and placement of the artificial valve. Once the artificial valve is in place, the catheter is removed from the body and the incision is closed.

Current TAVR approaches may also employ transesophageal echocardiogram (TEE) to visualize positioning and placement of the artificial valve. TEE is a type of echocardiogram that uses an ultrasound probe inserted through the throat and into the esophagus. It is performed to diagnose certain heart conditions and is also used to rule out blood clots in the heart prior to a procedure. Because the heart sits proximal to the esophagus, the TEE generally provides a clear and detailed imaging of the heart structure and function.

An example TAVR system is described in the article “A New Transcatheter Aortic Valve and Percutaneous Valve Delivery System” by John G. Webb, MD et al., Journal of the American College of Cardiology, Vol. 53, 2009:1855-8, the teachings of which are incorporated by references in their entirety.

Endovascular aneurysm repair is a procedure to treat an abdominal aortic aneurysm (AAA). The procedure is performed by inserting one or more graft components, which are typically folded and compressed within a delivery sheath, through the lumen of an access blood vessel, usually the common femoral artery. As with TAVR, endovascular aneurysm repair involves using X-ray imaging to guide the graft(s) into place.

A combined intravascular ultrasound (IVUS) and stent delivery device has previously been described in the article by Rieber, J., et al. (2005), “Application, Feasibility, and Efficacy of a Combined Intravascular Ultrasound and Stent Delivery System: Results from a Prospective Multicenter Trial.” Journal of Interventional Cardiology, 18:367-374, available at https://doi.org/10.1111/j.1540-8183.2005.00075.x. Another combined IVUS and stent delivery device has been described in the article by Eeckhout, E., et al. (2003), “Direct Stenting With a Combined Intravascular Ultrasound-Coronary Stent Delivery Platform: A Feasibility Trial,” Catheterization and Cardiovascular Interventions 59:451-454.

An IVUS imaging catheter including a balloon carrying a stent is described in WO/2002/007601A2, by Jomed Imaging Limited, titled “Ultrasonic imaging catheters.” A stent delivery catheter including an IVUS imaging transducer is described in WO/2002/064061A2, by JOMED GMBH, titled “Stent having a web structure and suitable for forming a curved stent.” Another stent delivery catheter including an IVUS imaging transducer is described in European Patent EP1279382A1, by JOMED NV, titled “Curved stent.”

An intravascular ultrasound (IVUS) ostial stent delivery system and method are described in U.S. Pat. No. 11,510,798 B1, by Kahlon. An integrated therapeutic imaging catheter and methods are described in US 2014/0276028 A1, by Stigall et al. A stent delivery device with an IVUS transducer on the tip of a delivery catheter is described in US 2023/0098512 A1, by Kahn. An ultrasound-guided delivery system for positioning/repositioning of transcatheter heart valves is described in US 2019/00152303 A1, by Kheradvar.

As best understood, none of the above prior approaches describe using position encoding readable by a position sensor of a delivery catheter.

Examples of systems and methods providing for position detection of endoluminal instruments are described in International Application No. PCT/US2021/072780, titled “Methods and Systems for Body Lumen Medical Device Location,” published as International Publication No. WO 2022/126101 A2, the entire teachings of which are incorporated herein by reference.

Examples of systems, devices, and methods for measuring relative displacement between at least two flexible elongate instruments within a body lumen are described in International Application No. PCT/US2023/064168, titled “Devices and Methods for Endoluminal Position Detection” published as International Publication No. WO 2023/173108 A1, the entire teachings of which are incorporated herein by reference. The provided systems and methods can enable improved position detection, including orientation and direction detection, of flexible elongate instruments disposed within a body lumen.

The attached drawings illustrate improvements to the systems devices, and methods provided in Intl. Pub. No. WO 2022/126101 A2, Intl. Pub. No. WO 2023/173108 A1, or both, as further described herein.

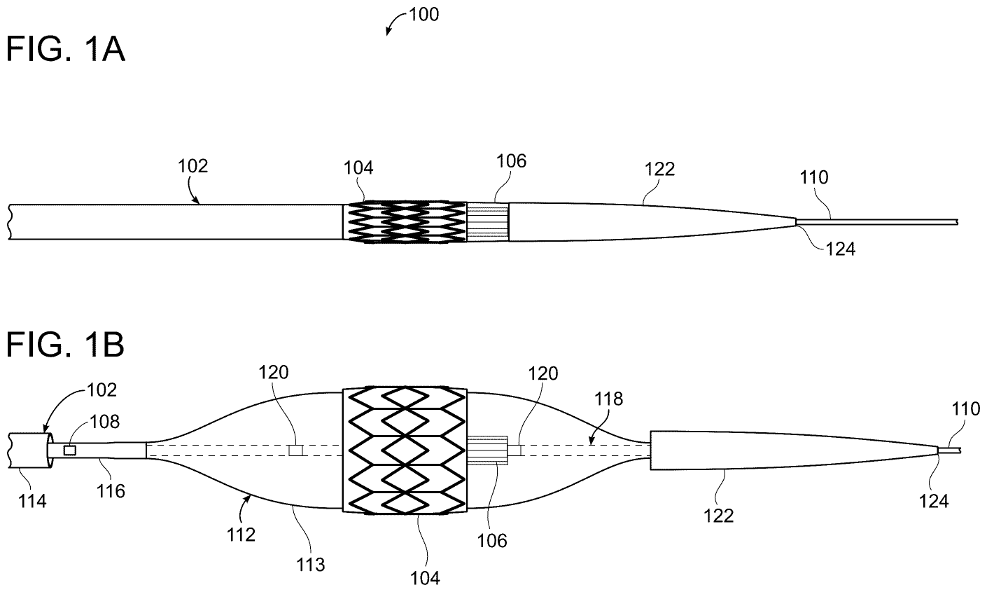

FIGS. 1A-1B illustrate a delivery system 100 for placement of an endoluminal device 104. The system 100 includes a delivery catheter 102 (e.g., a first flexible elongated instrument) comprising an imaging sensor 106 and a position sensor 108. The position sensor 108 is located at a known distance from the imaging sensor 106 and configured to sense position relative to an element, for example, a guidewire 110. The delivery catheter 102 is configured for mounting the endoluminal device 104, for example, a TAVR valve as illustrated. The endoluminal device 104 and the delivery catheter 102 may be provided separately, e.g., packaged separately, and then assembled during the procedure. The endoluminal device 104 (e.g., an aortic valve, a stent graft, a stent, an ultrasound heating device, an acoustic emitting device, a heating device, or a vessel cutting device etc.) can be mounted on the delivery catheter 102 and located at a known distance from the imaging sensor 106 or the position sensor 108.

The system 100 includes a deployment mechanism 112 to deploy the endoluminal device 104 in a body lumen. For example, as illustrated in FIG. 1B, the endoluminal device 104 is balloon-expandable and the deployment mechanism 112 includes a balloon membrane 113.

The delivery catheter 102 comprises an outer sheath 114 and an inner shaft 116 that is configured to move with respect to the outer sheath 114. In FIG. 1B, the outer sheath 114 is moved relative to the inner shaft 116 when compared to the configuration shown in FIG. 1A. The inner shaft 116 defines a lumen 118 that allows for the passage of the guidewire 110. The system 100 can further include one or more radiopaque imaging markers 120. At a distal end 124 of the delivery catheter 102, e.g., at a distal end of the inner shaft 116, a cone-shaped tip 122 is provided to facilitate insertion of the catheter into a body lumen. As illustrated, the guide wire 110 exits at a distal opening of the cone-shape tip.

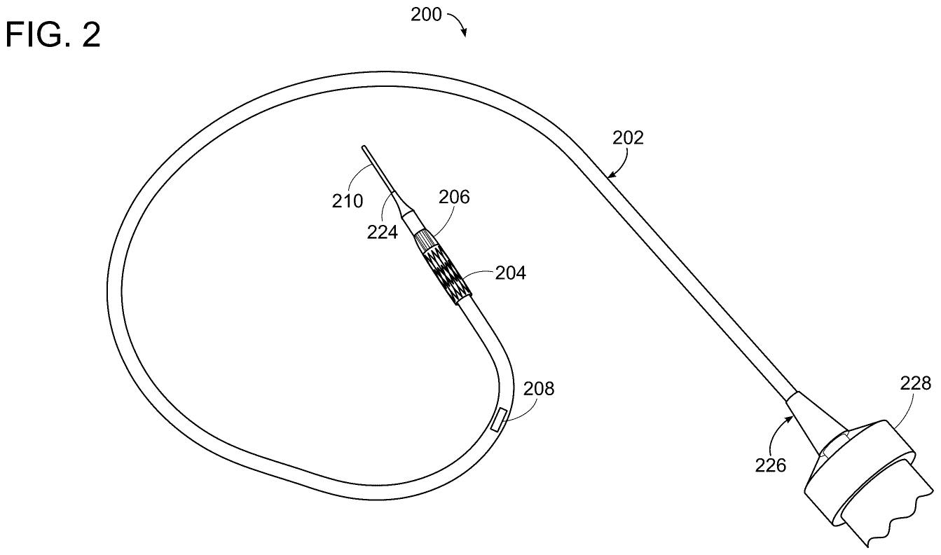

FIG. 2 illustrates another delivery system 200 including a delivery catheter 202 having a distal end 224 and a proximal end 226. The delivery catheter 202 can be configured to couple with a handle 228 at the proximal end 226 of the delivery catheter. The handle 228 can include an activation mechanism configured to activate the deployment mechanism, e.g. balloon deployment mechanism 112 in FIG. 1A. Circuitry to drive the imaging and position sensors and receive imaging and position signals can be housed in the handle 228. The handle may also include wireless communication circuitry to send and receive imaging data, position data, and other information related to the catheter system. As with delivery system 100, delivery system 200 further includes an imaging sensor 206, position sensor 208, and guidewire 210.

The system can be configured for intravascular imaging by including a suitable imaging sensor 106, 206 (e.g., an IVUS imaging sensor element or an OCT imaging sensor element). An IVUS imaging sensor typically includes an imaging transducer configured for rotation within the catheter. The system 100, 200 can include a suitable position sensor 108, 208 (e.g., an optical sensor) configured to detect position information encoding (e.g., markers encoding displacement, position, and/or orientation) of an element, such as a guidewire (e.g., a second flexible elongate instrument) disposed in a body lumen. The position sensor 108, 208 is fixedly engaged with the delivery catheter 102, 202, or at least a portion of the delivery catheter, to place the sensor at a defined distance from the imaging sensor 106, 206 and enable the position sensor 108, 208 to move together with the delivery catheter 102, 202 in the body lumen. In FIGS. 1A-1B, the position sensor 108 is fixedly engaged with the inner shaft 116 of the delivery catheter 102, whereby the distance from the imaging sensor 106 is defined (e.g., fixed). In FIG. 2, the position sensor 208 is fixedly engaged with an outer sheath or an inner shaft of the delivery catheter 202, whereby the distance from the imaging sensor 206 is defined (e.g., fixed or known).

As shown in the example configurations in the drawings, delivery catheters 102, 202 can be provided with a position sensor 108, 208 or reader (e.g., readers as shown in the example devices of FIGS. 12, 13, 14, 15, 32, 35, 36 of Intl. Pub. No. WO 2022/126101 A2), which can detect position encoding. For example, the position encoding readable by the position sensor can be on a guidewire 110, 210 (e.g., another flexible elongate instrument) disposed in a body lumen. Generally, the position encoding is on an element configured for relative movement with respect to the position sensor. In one example, the delivery catheter comprises an outer sheath (e.g., an outer shell) and an inner shaft that is movable relative to the outer sheath. One of the inner shaft or the outer sheath can be the element with the position code, while the other of the inner shaft or the outer sheath can include the position sensor. Also, in the example where the position code is on the guidewire, the position sensor can be positioned on the inner shaft of the catheter, for example, where the guidewire runs through a lumen of the inner shaft.

Various sensing modalities of the position sensor are contemplated. Example sensing modalities, including position sensors, and suitable encoding markers readable by the position sensors, are described in Intl. Pub. No. WO 2022/126101 A2, the teachings of which are incorporated herein by reference. For example, the position sensor can be an optical sensor, an electrical sensor, an electromagnetic senor, a mechanical sensor, an electromechanical sensor, a pressure sensor, a chemically-selective sensor, and/or a sonographic sensor. The sensing modality may be selected based on a particular application, a particular anatomical location, or other factors.

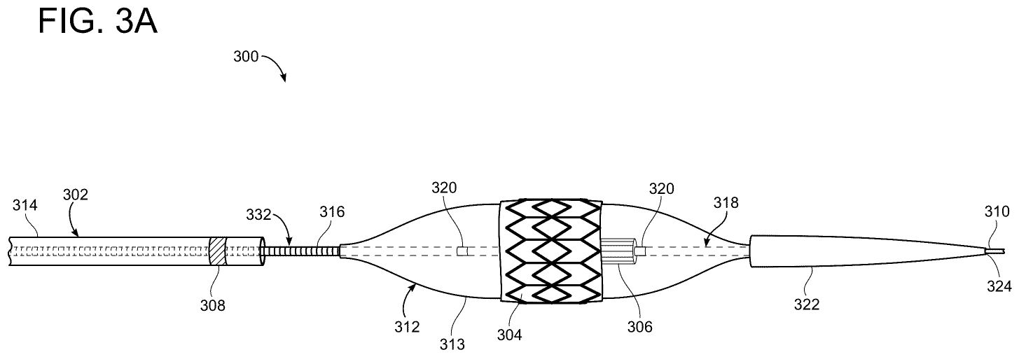

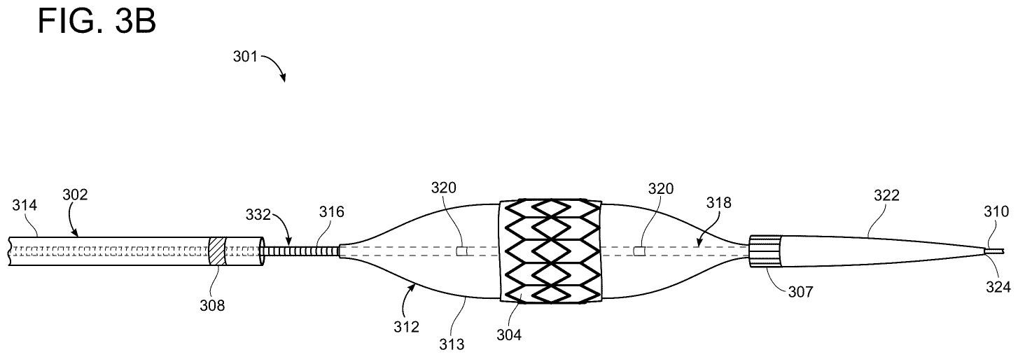

FIG. 3A illustrates an example delivery system 300 comprising a delivery catheter 302 that includes a slidable inner shaft 316 that includes position barcode 332 to be read by a position sensor 308 on the outer sheath 314 of the catheter. The barcode 332, the endoluminal device 304 (i.e., the replacement valve), and the (IVUS) imaging sensor 306 are all on the same slidable shaft 316. It will be understood that the position sensor 308 can alternatively be on the inner shaft 316, in which case the position barcode 332 would be on the outer sheath 314. As with the delivery system 100, the delivery system 300 includes a guidewire 310, a lumen 318 defined by the inner shaft 316 to receive the guidewire 310, a mechanism 312 to deploy an endoluminal device 304, e.g. a balloon membrane 313, and a cone-shaped tip 322 at a distal end 324. The position sensor 308 is fixedly engaged with the delivery catheter 302, e.g., the outer sheath 313 of the delivery catheter, to place the sensor at a defined distance from the imaging sensor 306. The distance is defined because the distance between the position bar code 332 and the imaging sensor 306 is defined (e.g., fixed), whereby the distance of the position sensor 308 from the imaging sensor 306 is determinable.

In FIG. 3A, the imaging sensor 306 is located inside the balloon membrane 313 of the deployment mechanism 312. Alternatively, FIG. 3B illustrates a system 301 similar to system 300 but with an imaging sensor 307 located outside the balloon membrane of the deployment mechanism 312, for example distally to the balloon 313. As illustrated, the imaging sensor 307 can be positioned at the cone-shaped tip 322 of the delivery catheter 302.

The system can optionally include a localization sensor or marker 320 (e.g., an imaging visible marker) that can enable registration of a system coordinate frame of reference with a coordinate frame of reference of another modality. The position (spatial) encoding markers 332 (or a position of the device as detected from the position encoding markers) can be spatially aligned with respect to one or more imaging-visible markers 320 of the system. The spatial alignment can provide for registration (e.g., automatic registration, co-registration) of a coordinate frame of reference of the system with an imaging frame of reference.

Example techniques for establishing a reference coordinate system based on a plurality of imaging markers, receiving imaging information (e.g., diagnostic scan information) at a plurality of locations of the first or second flexible elongate instrument, for example the outer sheath 314 and inner shaft 316 of FIG. 3A, and correlating the information with the imaging markers are described in Intl. Pub. No. WO 2022/126101 A2.

The system and methods of the present described herein have many advantages. Because the system includes an image sensor mounted on the delivery catheter to image the heart tissue locally, e.g., from within the heart, there is no need to employ TEE. This can reduce or eliminate time and cost (equipment, personnel) associated with the TEE procedure, thereby reducing overall time and cost of the procedure. Further, by not using TEE, the need for general anesthesia can be avoided.

Including an imaging sensor, e.g. sensors 306 or 307, and a position sensor 308 in the delivery catheter may increase the overall diameter of the catheter 302, but such increase is expected to be minimal. The catheter 302 may include one or more additional lumens, e.g. additional lumens beyond lumen 318, for sensor leads providing connections between the sensors and control circuitry. It is expected that the catheter's crossing profile is still determined by the endoluminal device 304 (e.g., the implant, heart valve, stent, etc.) mounted on the catheter 302.

The IVUS transducer can be detachably mounted to the catheter shaft. The TAVR catheter shaft typically has a relatively large diameter, which makes this type of mounting possible and can make the IVUS transducer reusable to reduce cost.

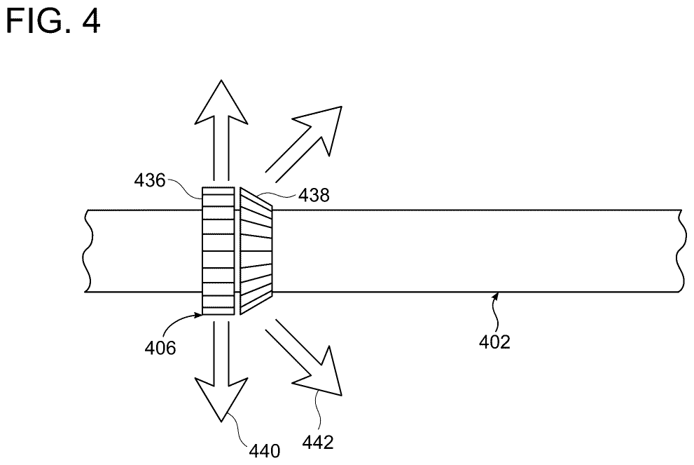

FIG. 4 illustrates another example delivery catheter 402 with an imaging sensor 406 that has side-looking (side-firing) capability 440 and forward-looking (forward-firing) capability 442. Here, the imaging sensor 406 includes two intravascular ultrasound (IVUS) ring array transducers, 436 and 438, including at least one of the ring array transducers having the forward-looking capability. As illustrated, ring array transducer 436 is configured to have a side-looking 440 (e.g. axial) component and ring array transducer 438 is configured at an angle to have a forward-looking 442 (e.g. longitudinal) component. The forward-looking 442 component can be used to detect blood flow, e.g., via pulsed doppler velocimetry. For example, blood flow may be measured as the product of blood vessel cross-sectional area (obtained via IVUS imaging of the vessel) and mean velocity derived from pulsed doppler velocimetry. Although the imaging sensor 406 having two ring transducer is shown positioned on a shaft of the catheter 402, the sensor may be used with other delivery catheters described herein, such as, for example, the inner shaft 316 of catheter 302 of system 300 illustrated in FIG. 3A. Furthermore, the sensor 406 can also be positioned or integrated into a cone-shaped tip, such as tip 322 of system 301.

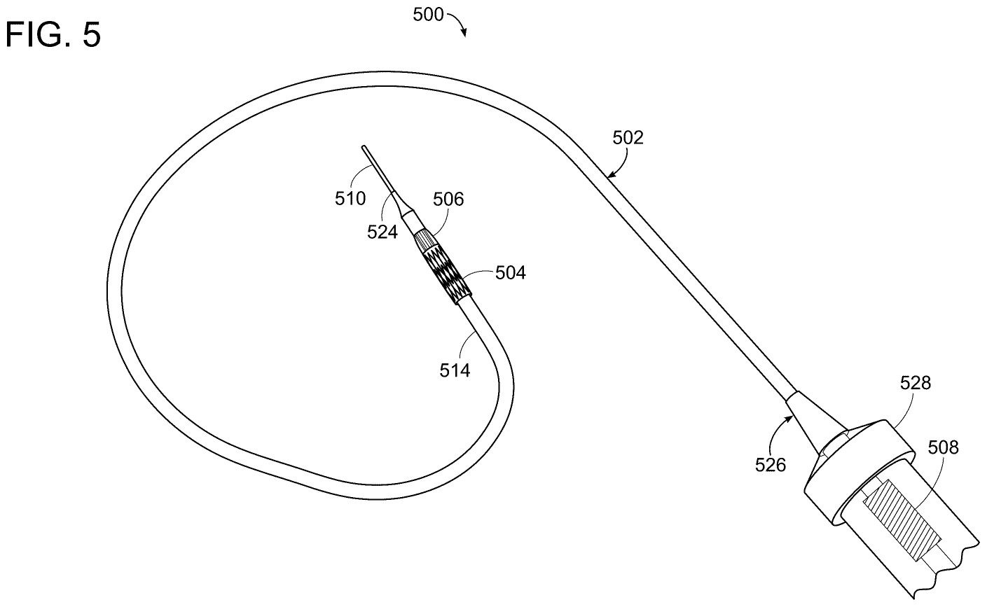

FIG. 5 illustrates another example delivery system 500 in which a position sensor 508 is positioned in a hub 528 at a proximal end 526 of the delivery catheter 502. The hub 528 may be in the form of a handle, as illustrated, or may be integrated into a handle. When the position sensor 508 is in the hub/handle 528, the element with the position code (e.g., position code 332 in FIG. 3A) can be any element that runs through the hub/handle, such a guidewire 510, an inner shaft (not shown in FIG. 5 but see, e.g., inner shaft 316 in FIG. 3A) of the delivery catheter 502, or an outer sheath 514 of the delivery catheter 502. Displacement measurement when measured at the hub/handle 528 may not be as accurate as when measured at or near the distal end 524 of the delivery catheter 502. The inaccuracy when measuring at the hub/handle 528 can be due to slack between the moving parts of the delivery system, such as the guidewire 510, the inner shaft (not shown), the outer sheath 514, or other parts of the delivery catheter 502.

The systems and devices illustrated in FIGS. 1-5, such as the systems 100, 200, 300, 301, 500, and, for example, delivery catheters 102, 202, 302, 502 including imaging sensors 106, 206, 306, 307, 406, 506 and position sensors 108, 208, 308, 508 configured to read a position code, e.g., position code 332, can be used to place an endoluminal device, e.g., device 104, 204, 304, or 504. Methods for the placement of an endoluminal device using any of the previously described embodiments of a delivery system may include: 1) guiding the a delivery catheter with a mounted endoluminal device through a body lumen to an anatomical location; 2) placing the endoluminal device at the anatomical location; and 3) confirming placement of the endoluminal device at the anatomical location using an imaging sensor, a position sensor, or both an imaging and a position sensor. Further, the endoluminal device can be mounted at a known distance from the imaging sensor or the position sensor.

With reference to FIGS. 1A and 1B, the delivery catheter 102 of system 100 can first be guided to the proximity of the anatomical location using a guidewire 110 and X-ray imaging with radiopaque imaging markers 120. Intravascular imaging, for example, IVUS or OCT, can be acquired using imaging sensor 106 while tracking displacement distance of an inner shaft 116 of the catheter 102 during imaging with respect to a stationary element with a position code, e.g., the guidewire 110, using position sensor 108. Features acquired from intravascular imaging may be further correlated with features from X-ray imaging of radiopaque makers 120. The inner shaft 116 of catheter 102 can then re-traverse to the anatomical location of treatment identified by intravascular imaging using the position code and position sensor 108 and deploy an endoluminal device 104 using a mechanism 112, including a balloon membrane 113. Placement of the endoluminal device can be confirmed through re-imaging of at least a part of the body lumen with the imaging sensor 106, potentially under guidance by position sensor 108.

Positioning the imaging sensor 106 at a known distance from the position sensor 108 can be useful for precise correlation of anatomical features with distance traveled along a length of a body lumen by moving the inner shaft 116 with position sensor 108 along the stationary element with the position code, e.g., the guidewire 110. In current practices, the displacement of an imaging sensor can be measured by an actuator external to a human body controlling a delivery catheter; however, motor position readings for an actuator external to a human body can be inaccurate due to reasons including the inherent elasticity of physiological vessels and tissues. Positioning the endoluminal device 104 at a known distance from the imaging sensor 106 or the position sensor 108 can be useful for precision deployment of the endoluminal device 104, which can be positioned proximal or distal to the imaging sensor along the catheter, to an anatomical site imaged using the imaging sensor 106 or X-ray imaging.

Alternatively, the embodiments illustrated in FIGS. 3A and 3B include a position sensor 308 on an outer sheath 314 of catheter 302 and a position code 332 on an inner shaft 316. Displacement of an imaging sensor 306, 307 and endoluminal device 304 can be determined by relative displacement of the inner shaft 316 with respect to the outer sheath 314 by reading a position code 332 disposed on the inner shaft 316 by a position sensor 308 on the outer sheath. As with system 100, positioning the imaging sensor 306, 307 at a known distance from the position sensor 308 can be useful for precise correlation of anatomical features with distance traveled along a length of a body lumen by moving the inner shaft 316 with the imaging sensor 306, 307 and position code 332 while holding the outer sheath 314 with the position sensor 308 stationary. Positioning the endoluminal device 304 at a known distance from the imaging sensor 306, 307 or the position sensor 308 can be useful for precision deployment of the endoluminal device 304, which can be positioned proximal or distal to the imaging sensor 306, 307, to an anatomical site imaged using the imaging sensor 106 or X-ray imaging.

FIG. 5 illustrates another embodiment in which a position sensor 508 is positioned in a hub/handle 528 of system 500, which can provide more space for electronic components without increasing the diameter of a delivery catheter 502.

Methods for the placement of an aortic valve using the systems and devices described herein may include: 1) guiding a delivery catheter with an imaging sensor, an element with a position code, a position sensor at a known distance from the imaging sensor, and an aortic valve mounted on the catheter at a known distance from the imaging sensor or the position sensor, to a heart; and 2) placing the aortic valve in the heart at a location confirmed by the imaging sensor, the position sensor, or a combination thereof. Such methods would be similar to those previously described for embodiments of a delivery system for an endoluminal device.

Optionally the position code element can be integrated in or disposed on the guidewire, such as in the embodiment illustrated in FIG. 1A-1B, the inner shaft, such as in the embodiment illustrated in FIG. 3A-3B, or on the outer sheath, such as in the embodiment illustrated in FIG. 5. Further, the position sensor can be located between the aortic valve and the proximal end of the delivery catheter.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A delivery system for placement of an endoluminal device, the system comprising:
a delivery catheter comprising an imaging sensor and a position sensor, the position sensor located at a known distance from the imaging sensor and configured to sense position relative to an element with position code readable by the position sensor,
the delivery catheter configured for mounting an endoluminal device on the delivery catheter at a known distance from the imaging sensor or the position sensor.

2. The system of claim 1, wherein the imaging sensor includes an intravascular ultrasound (IVUS).

3. The system of claim 2, wherein the imaging sensor includes an optical coherence tomography (OCT) sensor.

4. The system of claim 1, wherein the imaging sensor is located between the endoluminal device and a distal end of the delivery catheter.

5. The system of claim 1, wherein the element is a guidewire, and the position sensor is configured to read the position code from the guidewire.

6. The system of claim 1, wherein the position sensor is located between the endoluminal device and a proximal end of the delivery catheter.

7. The system of claim 1, wherein the endoluminal device includes one or more of the following: an aortic valve, a stent graft, or a stent.

8. The system of claim 1, wherein the endoluminal device includes one or more of the following: an ultrasound heating device, an acoustic emitting device, a heating device, or a vessel cutting device.

9. The system of claim 1, wherein the catheter includes a lumen for receiving a guidewire, the lumen extending through the imaging sensor and the position sensor.

10. The system of claim 1, further comprising the element with position code readable by the position sensor.

11. The system claim 1, further comprising a deployment mechanism to deploy the endoluminal device in a body lumen.

12. The system of claim 11, wherein the endoluminal device is balloon-expandable and the deployment mechanism includes a balloon membrane.

13. The system of claim 12, wherein the imaging sensor is located inside the balloon membrane.

14. The system of claim 11, further comprising a handle at a proximal end of the delivery catheter, the handle including an activation mechanism configured to activate the deployment mechanism.

15. The system of claim 1, further comprising the endoluminal device.

16. The system of claim 15, wherein the endoluminal device is mounted on the delivery catheter and located at a known distance from the imaging sensor or the position sensor.

17. The system of claim 1, wherein the delivery catheter comprises an outer sheath and an inner shaft movable relative to the outer sheath, one of the inner shaft or the outer sheath being the element with the position code, the other of the inner shaft or the outer sheath including the position sensor.

18. The system of claim 17, wherein the imaging sensor is positioned at or on the inner shaft.

19. The system of claim 18, wherein the imaging sensor includes an intravascular ultrasound (IVUS) transducer detachably mounted to the inner shaft.

20. The system of claim 1, wherein the imaging sensor has side-looking capability and forward-looking capability.

21. The system of claim 20, wherein the imaging sensor comprises two intravascular ultrasound (IVUS) ring array transducers, at least one of the ring array transducers having the forward-looking capability.

22. The system of claim 1, wherein the position sensor is in a handle at a proximal end of the delivery catheter.

23. The system of claim 22, wherein the element with the position code is a guidewire, an inner shaft of the delivery catheter, or an outer sheath of the delivery catheter.

24. The system of claim 1, wherein the position sensor is an optical sensor, an electrical sensor, an electromagnetic senor, a mechanical sensor, an electromechanical sensor, a pressure sensor, a chemically-selective sensor, and/or a sonographic sensor.

25. The system of claim 1, wherein the delivery catheter includes one or more imaging markers, wherein spatial alignment of the position code with respect to one or more imaging markers can enable registration of a coordinate frame of reference of the system with a frame of reference of an imaging system.

26. A method for placement of an endoluminal device using the system of claim 1, the method comprising:
guiding the delivery catheter and endoluminal device mounted on the delivery catheter through a body lumen to an anatomical location;
placing the endoluminal device at the anatomical location; and
with the imaging sensor, the position sensor, or both the imaging sensor and the position sensor, confirming placement of the endoluminal device at the anatomical location.

27. The method of claim 26, further including mounting the endoluminal device on the delivery catheter at a known distance from the imaging sensor or the position sensor.

28. A method for placement of an aortic valve, the method comprising:
guiding a delivery catheter through a blood vessel to a heart, the delivery catheter comprising an imaging sensor and a position sensor, the position sensor located at a known distance from the imaging sensor and configured to sense position relative to an element with a position code readable by the position sensor, an aortic valve mounted on the catheter and located at a known distance from the imaging sensor or the position sensor; and
placing the aortic valve in the heart at a location confirmed by the imaging sensor, the position sensor, or both the imaging sensor and the position sensor.

29. The method of claim 28, wherein the element is a guidewire, and the position sensor is configured to read the position code from the guidewire.

30. The method of claim 28, wherein the element is an inner shaft of the delivery catheter, and the position sensor is configured to read the position code from the inner shaft.

31. The method of claim 28, wherein the element is an outer sheath of the delivery catheter, and the position sensor is configured to read the position code from the outer sheath.

32. The method of claim 28, wherein the position sensor is located between the aortic valve and a proximal end of the delivery catheter.
